(19) United States
(12) Patent Application Publication
(10) Pub. No.: US 2008/0305118 A1
(43) Pub. Date: Dec. 11, 2008

(54) Treatment Of Type 1 Diabetes With Inhibitors Of Macrophage Migration Inhibitory Factor

(75) Inventor: Yousef Al-Abed, Locust Valley, NY (US)

Correspondence Address: AMSTER, ROTHSTEIN & EBENSTEIN LLP, 90 PARK AVENUE, NEW YORK, NY, 10016, US

(73) Assignee: The Feinstein Institute for Medical Research, Manhasset, NY (US)

(21) Appl. No.: 10/594,641

(22) PCT Filed: Mar. 29, 2005
(86) PCT No.: PCT/US2005/010521

§ 371 Date: Mar. 28, 2008

Related U.S. Application Data
(60) Provisional application No. 60/557,169, filed on Mar. 29, 2004.

(51) Int. Cl. A61K 39/395; A61K 38/02; A61K 31/713; C12Q 1/02; C12Q 1/68; A61P 3/10; A61K 31/7052

(52) U.S. Cl. 424/173.1; 514/2; 514/44; 435/29; 435/6

(57) ABSTRACT

Methods of treating a mammal having type 1 diabetes or a risk for type 1 diabetes are provided. The methods comprise administering to the mammal a pharmaceutical composition comprising an agent that inhibits MIF in the mammal. Also provided are methods of evaluating whether a compound is useful for preventing or treating type 1 diabetes. The methods comprise determining whether the compound inhibits a macrophage migration inhibitory factor (MIF) in a mammal, then, if the compound inhibits the MIF, determining whether the compound inhibits development of type 1 diabetes.

### CROSS-REFERENCE TO RELATED APPLICATION

This application claims the benefit of U.S. Provisional Application No. 60/557,169, filed Mar. 29, 2004.

### STATEMENT REGARDING FEDERALLY FUNDED RESEARCH OR DEVELOPMENT

The U.S. Government has a paid-up license in this invention and the right in limited circumstances to require the patent owner to license others on reasonable terms as provided for by the terms of Grant No. DK064233-01 awarded by The National Institutes of Health.

### BACKGROUND OF THE INVENTION

(1) Field of the Invention

The present invention generally relates to diabetes treatment. More specifically, the invention is directed to the use of inhibitors of macrophage migration inhibitory factor for treatment or prevention of type 1 diabetes.

(2) Description of the Related Art

### REFERENCES

1. Dahlquist G: The aetiology of type 1 diabetes: an epidemiological perspective. Acta Paediatr (Suppl. October) 425:5-10, 1998
2. Like A A, Rossini A A: Streptozotocin-induced pancreatic insulitis, a new model of diabetes mellitus. Science 193:415-417, 1976
3. Kolb H: IDDM: Lessons from the low-dose streptozotocin model in mice. Diabetes Rev 1:116-126, 1993
4. Sandberg J O, Andersson A, Eizirik D L, Sandier S: Interleukin-1 receptor antagonist prevents low dose streptozotocin induced diabetes in mice. Biochem Biophys Res Commun, 202:543-548, 1994
5. Herold K C, Vezys V, Sun Q, Viktora D, Seung E, Reiner S, Brown D R: Regulation of cytokine production during development of autoimmune diabetes induced with multiple low doses of streptozotocin. J Immunol 156:3521-3527, 1996
6. Holdstad M, Sandler S: A transcriptional inhibitor of TNF-α prevents diabetes induced by multiple low-dose streptozotocin injections in mice. J Autoimmun 16:441-447, 2001
7. Nicoletti F, Di Marco R, Papaccio G, Cognet I, Gomis R, Bernardini R, Sims J E, Shoenifeld Y, Bendzen K: Essential pathogenic role of endogenous IL-18 in murine diabetes induced by multiple low doses of streptozotocin. Prevention of hyperglycemia and insulitis by a recombinant IL-18-binding protein: Fc construct. Eur J Immunol 33:2278-2286, 2003
8. Rabinovitch A, Suarez-Pinzon W L: Cytokines and their roles in pancreatic islet β-cell destruction and insulin-dependent diabetes mellitus. Biochem Pharmacol 55:1139-1149, 1998
9. Campbell L, Kay T W H, Oxbrow L, Harrison L C: Essential role for interferon-γ and interleukin-6 in autoimmune insulin-dependent diabetes in NOD/Whei mice. J Clin Invest 87:739-742, 1991
10. Nicoletti F, Zaccone P, Di Marco R, Magro G, Grasso S, Stivala F, Calori G, Mughini L, Meroni P L, Garotta G: Paradoxical antidiabetogenic effect of γ-interferon in DP-BB rats. Diabetes 47:32-38, 1998
11. Yang X D, Tisch R, Singer S M, Cao Z A, Liblau R S, Schreiber D, McDevitt H O: Effect of tumor necrosis factor alpha on insulin-dependent diabetes mellitus in NOD mice. I. The early development of autoimmunity and the diabetogenic process. J Exp Med 180:995-1004, 1994
12. Winter W E, Schatz D: Prevention strategies for type 1 diabetes mellitus. Biodrugs 17:39-64, 2003
13. Bucala R: MIF rediscovered: cytokine, pituitary hormone, and glucocorticoid-induced regulator of the immune response. FASEB J 10:1607-1613, 1996
14. Metz C N, Bucala R: Role of macrophage migration inhibitory factor in the regulation of the immune response. Adv Immunol 66:197-223, 1997
15. Calandra T, Echtenacher B, Le Roy D, Pugin J, Metz C N, Hultner L, Heumann D, Mannel D, Bucala R, Glauser M P: Protection from septic shock by neutralization of macrophage migration inhibitory factor. Nat Med 6:164-170, 2000
16. De Yong Y P, Abadia-Molina A C, Satoskar A R, Clarke K, Rietdijk S T, Faubiom W A, Mizoguchi E, Metz C N, Al Sahli M, Ten Hove T, Keates A C, Lubetsky J B, Farrell R J, Michetti P, Van Deventer S J, Lolis E, David J R, Bhan A K, Terhorst C: Development of chronic colitis is dependent on the cytokine MIF. Nat Immunol 2:1061-1066, 2001
17. Denkinger C M, Denkinger M, Kort J J, Metz C, Forsthuber T G: In vivo blockade of macrophage migration inhibitory factor ameliorates acute experimental autoimmune encephalomyelitis by impairing the homing of encephalitogenic T cells to the central nervous system. J Immunol 170:1274-1282, 2003
18. Bojunga J, Kusterer K, Bacher M, Kurek R, Usadel K-H, Renneberg H: Macrophage migration inhibitory factor and development of type-1 diabetes in non-obese diabetic mice. Cytokine 21:179-186, 2003
19. Lue H, Kleemann R, Calandra T, Roger T, Bernhagen J: Macrophage migration inhibitory factor (MIF): mechanisms of action and role in disease. Microbes and Infection 4:449-460, 2002
20. Lubetsky J B, Dios A, Han J, Aljabari B, Ruzsiccska B, Mitchell R, Lolis E, Al Abed Y: The tautomerase active site of macrophage migration inhibitory factor is a potential target for discovery of novel anti-inflammatory agents. J Biolog Chem 277:24976-24982, 2002
21. Maksimovic-Ivanic D, Trajkovic V, Miljkovic D J, Mostarica Stojkovic M, Stosic-Grujicic S. Down-regulation of multiple low dose streptozotocin-induced diabetes by mycophenolate mofetil. Clin Exp Immunol 129:214-223, 2002
22. Stosic-Grujicic S, Maksimovic D, Badovinac V, Samardzic T, Trajkovic V, Lukic M, Mostarica Stojkovic M: Antidiabetogenic effect of pentoxifylline is associated with systemic and target tissue modulation of cytokines and nitric oxide production. J Autoimmun 16:47-58, 2001
23. Versteeg H H, Nijhuis E, Van Den Brink G R, Evertzen M, Pynaert G N, Van Deventer S J, Coffer P J, Peppelebosch M P: A new phosphospecific cell-based ELISA for p42/p44 mitogen-activated protein kinase (MAPK), p38 MAPK, protein kinase B and cAMP-response-element-binding protein. Biochem J 350:717-722, 2000
24. Lan H Y, Mu W, Yang N, Meinhardt A, Nikolic-Paterson D J, Ng Y Y, Bacher M, Atkins R C, Bucala R: De novo renal expression of macrophage migration inhibitory factor during the development of rat crescentic glomerulonephritis. Am J Pathol 149:1119-1127, 1996
25. Kunt T, Forst T, Fruh B, Flohr T, Schneider S, Harzer O, Pfutzner A, Engelbach M, Lobig M, Beyer J: Binding of monocytes from normolipidemic hyperglycemic patients with type 1 diabetes to endothelial cells is increased in vitro. Exp Clin Endocrinol Diabetes 107:252-256, 1999
26. Hatamori N, Yokono K, Hayakawa M, Taki T, Ogawa W, Nagata M: Anti-interleukin-2 receptor antibody attenuates low-dose streptozotocin-induced diabetes in mice. Diabetologia 33:266-271 1990
27. Lan H Y, Bacher M, Yang N, Mu W, Nikolic-Paterson D J, Metz C, Meinhardt A, Bucala R, Atkins R C: The pathogenic role of macrophage migration inhibitory factor in immunologically induced kidney disease in the rat. J Exp Med 185:1455-1465, 1997
28. Juttner S, Bernhagen J, Metz C N, Rollinghoff M, Bucala R, Gessner A: Migration inhibitory factor induces killing of Leishmania major by macrophages: dependence on reactive nitrogen intermediates and endogenous TNF-α. J Immunol 161:2383-2390, 1998
29. Bozza M, Satoskar A R, Lin G, Humbles A A, Gerard C, David J R: Targeted disruption of migration inhibitory factor gene reveals its critical role in sepsis. J Exp Med 189:341-346, 1999
30. Calandra T, Spiegel L A, Metz C N, Bucala R: Macrophage migration inhibitory factor is a critical mediator of the activation of immune cells by exotoxins of Gram-positive bacteria. Proc Natl Acad Sci USA 95:11383-11388, 1998
31. Mikulowska A, Metz C N, Bucala R, Holmdahl R: Macrophage migration inhibitory factor is involved in the pathogenesis of collagen type II-induced arthritis in mice. J Immunol 158:5514-5517, 1997
32. Waeber G, Calandra T, Roduit R, Haefliger J-A, Bonny C, Thompson N, Thorens B, Temler E, Meinhardt A, Bacher M, Metz C N, Nicod P, Bucala R: Insulin secretion is regulated by the glucose-dependent production of islet β cell macrophage migration inhibitory factor. Proc Natl Acad Sci USA 94:4782-4787, 1997
33. Bacher M, Metz C N, Calandra T, Mayer K, Chesney J, Lohoff M, Gemsa D, Donnely T, Bucala R: An essential regulatory role for macrophage migration inhibitory factor in T-cell activation. Proc Natl Acad Sci USA 93:7849-7854, 1996
34. Lub M, Van Kooyk Y, Figdor C G: Competition between lymphocyte function-associated antigen (CD11a/CD18 and MAC-1 (CD11b/CD18) for binding to intercellular adhesion molecule-1 (CD54). J Leukoc Biol 59:648-655, 1996
35. Bernhagen J, Calandra T, Bucala R: The emerging role of MIF in septic shock and infection. Biotherapy 8:123-127, 1994
36. Wachlin G, Augstein P, Schroder D, Kuttler B, Kloting I, Heinke P, Schmidt S: IL-1beta, IFN-gamma and TNF-alpha increase vulnerability of pancreatic beta cells to autoimmune destruction. J Autoimmun 20:303-312, 2003
37. Nicoletti F, Zaccone P, Di Marco R, Lunetta M, Magro G, Grasso S, Meroni P, Garotta G: Prevention of spontaneous autoimmune diabetes in diabetes-prone BB rats by prophylactic treatment with anti-rat interferon-γ antibody. Endocrinology 138:281-288, 1997
38. Calandra T, Bernhagen J, Metz C N, Spiegel L A, Bacher M, Donelly T, Cerami A, Bucala R: MIF as a glucocorticoid-induced modulator of cytokine production. Nature 377:68-71, 1995
39. Debets R, Savelkoul H F J: Cytokine antagonists and their potential therapeutic use. Immunol Today 15:455-458, 1994
40. Bozza M, et al: Targeted disruption of migration inhibitory factor gene reveals its critical role in sepsis. J. Exp. Med. 189:341-346, 1999

Type 1 diabetes mellitus (type 1 DM) is a multifactorial syndrome caused by the lack of endogenous insulin, thought to be due to an immune attack mediated by autoreactive T cells and macrophages against pancreatic β-cells. The disease afflicts approximately 4 million people in North America and epidemiological data concur that the incidence and thus the prevalence of the disease is increasing worldwide (1). Extensive research efforts have greatly expanded understanding of disease pathogenesis, and have revealed a critical role for several pro-inflammatory mediators. However, no effective anti-inflammatory therapeutics have been approved for the clinical management of type 1 DM. Several animal models of the disease have enhanced understanding of the molecular events that underlie the pathogenesis of diabetes. Multiple low doses of streptozotocin to susceptible strains of mice, for example, induces a diabetic condition with many of the hallmarks of human type 1 DM. Clinical and histoimmunological similarities include the development of hyperglycemia associated with infiltration of the pancreatic islets by T lymphocytes and macrophages (insulitis) (2,3). Proinflammatory cytokines, including interleukin (IL)-1β, interferon (IFN)-γ, tumor necrosis factor (TNF)-α and IL-18 play important roles in the development of streptozotocin-induced diabetes (4-7). However, administration of either recombinant IL-1β, IFN-γ, or TNF-α, or specific inhibitors of their activity, have complex and often contradictory effects on disease development and/or course, depending on animal model used, as well as on timing of administration (8-11).

The key pathogenic role played by the immune system in the pathogenesis of type 1 DM has recently focused much attention on identifying immunotherapeutical approaches that may allow halting or delaying β-cell destruction in prediabetic individuals or in those patients with newly diagnosed disease (12). Macrophage migration inhibitory factor (MIF) is a critical cytokine in local and systemic inflammation, but its role in diabetes has not been explored thoroughly. MIF is a pleiotropic cytokine produced during immune responses by activated T cells, macrophages and a variety of nonimmune cells (13,14). It acts as a critical mediator of host defense, and is being explored as a therapeutic target in septic shock as well as chronic inflammatory and autoimmune diseases (15-17). Elevated MIF gene expression has been detected in spontaneously non-obese diabetic (NOD) mice (18), but its importance in the pathogenesis of type 1 DM is unclear.

There is thus a need for further investigation into the precise role and interactions of cytokines, in particular MIF, in type 1 diabetes. The present invention addresses that need.

### SUMMARY OF THE INVENTION

Accordingly, the inventors have discovered that inhibition of macrophage migration inhibitory factor (MIF) attenuates type 1 diabetes.

Thus, in some embodiments, the invention is directed to methods of treating a mammal having type 1 diabetes or at risk for type 1 diabetes. The methods comprise administering to the mammal a pharmaceutical composition comprising an agent that inhibits a macrophage migration inhibitory factor (MIF) in the mammal. In these embodiments, the agent is a polypeptide or a polynucleotide.

The present invention is also directed to other methods of treating a mammal having type 1 diabetes or at risk for type 1 diabetes. These methods also comprise administering to the mammal a pharmaceutical composition comprising an agent that inhibits a macrophage migration inhibitory factor (MIF) in the mammal. In these embodiments, the agent is an organic molecule comprising the following structure I or II

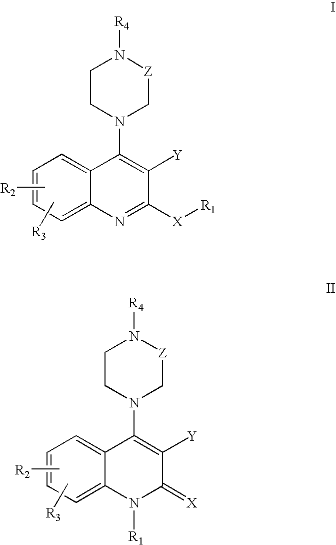

The invention is additionally directed to methods of evaluating whether a compound is useful for preventing or treating type 1 diabetes. The methods comprise, (a) determining whether the compound inhibits a macrophage migration inhibitory factor (MIF) in a mammal, then, if the compound inhibits the MIF, (b) determining whether the compound inhibits development of type 1 diabetes.

In additional embodiments, the invention is directed to kits comprising (a) a pharmaceutical composition comprising an agent that inhibits a macrophage migration inhibitory factor (MIF) in the mammal, where the agent is a polypeptide or a polynucleotide, and (b) instructions for administering the composition to the mammal. In these embodiments, the mammal has type 1 diabetes or is at risk for type 1 diabetes.

In further embodiments, the invention is directed to other kits comprising (a) a pharmaceutical composition comprising an agent that inhibits a macrophage migration inhibitory factor (MIF) in the mammal, and (b) instructions for administering the composition to the mammal. In these embodiments, the mammal has type 1 diabetes or is at risk for type 1 diabetes. The pharmaceutical composition of these embodiments is an organic molecule comprising the following structure I or II

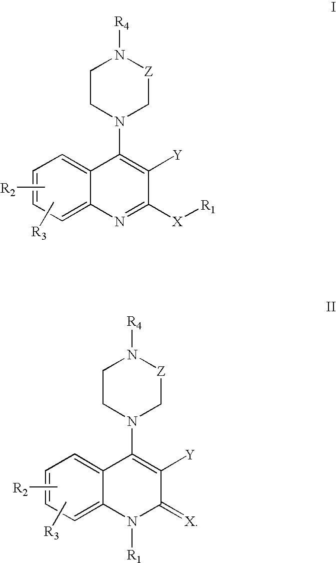

The invention is also directed to the use of an agent that inhibits a macrophage migration inhibitory factor (MIF) in the mammal, where the agent is a polypeptide or a polynucleotide, for the manufacture of a medicament for the treatment of a mammal having type 1 diabetes or at risk for type 1 diabetes.

Additionally, the invention is directed to the use of an agent that inhibits a macrophage migration inhibitory factor (MIF) in the mammal for the manufacture of a medicament for the treatment of a mammal having type 1 diabetes or at risk for type 1 diabetes. In these embodiments, the agent is an organic molecule comprising the following structure I or II

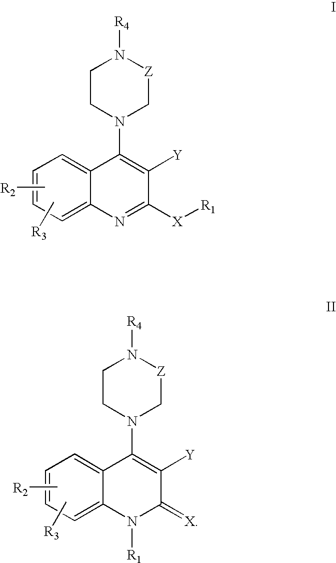

### BRIEF DESCRIPTION OF THE DRAWINGS

FIG. 1 is micrographs and graphs demonstrating elevated MIF protein expression in pancreas and peritoneal cells of diabetic mice. Panels A-C are light immunomicrographs of pancreata. MIF is weakly expressed by islet cells of non-diabetic control mice (Panel A); in pancreatic islets of day 10 diabetic mice, there is massive mononuclear cell (MNC) infiltration and MIF expression is markedly up-regulated (Panel B); negative staining in a serial section of diabetic mice islet (Panel C). Panel D shows a graph of quantitative image analysis showing different stages of MIF expression by islet cells from diabetic versus non-diabetic control mice. Shown are the mean±SD of the percentage of MIF⁺ cells per islet (n=3 mice per group). Panel E is a graph of quantitative analysis of intracellular expression of MIF protein in peritoneal cells of non-diabetic mice (Control), MLD-STZ diabetic mice (STZ), and MLD-STZ diabetic mice treated with anti-MIF antibody (STZ-αMIF), measured by cell-based ELISA performed with MIF-specific antibodies, as described in Materials and Methods of the Example. The results are presented as fold increase of control absorbance value (OD 492 nm 0.687±0.013). *p<0.05 refers to otherwise untreated MLD-STZ diabetic animals.

FIG. 2 is graphs of experimental results demonstrating that MIF blockade with anti-MIF antibody and ISO-1 suppress the development of hyperglycemia and insulitis. Blood glucose levels were determined in C57B1/6 mice (Panel A) and CBA/H mice (Panel B) starting at the beginning of the STZ treatment and continued through weekly measurements. Animals received MLD-STZ injections and were treated with either vehicle (STZ), non-immune rabbit IgG (STZ-IgG), anti-MIF IgG (STZ-αMIF), or ISO-1 (STZ-ISO). Histopathology analysis of pancreata from C57B1/6 mice (Panel C) and CBA/H mice (Panel D) are presented as insulitis score, as described in as described in Materials and Methods of the Example. *p<0.05 refers to corresponding STZ or STZ-IgG animals.

FIG. 3 is graphs of experimental results demonstrating that neutralization of MIF activity reduces splenocyte proliferation and adherence. Panel A shows ³H-thymidine incorporation, as a measure of cell proliferation, as determined in SMNC isolated from mice untreated with STZ (control), treated with STZ and vehicle (STZ), with STZ and non-immune rabbit IgG (STZ-IgG), STZ and anti-MWF IgG (STZ-αMIF), or STZ and ISO-1 (STZ-ISO). Panel B shows adhesion to plastic surface, or L929 fibroblast, as determined for SMNC isolated from the same groups of mice. The results are presented as fold increase of control adhesion to plastic surface, or to L929 fibroblasts (O.D. 570 nm 0.316±0.018 and 0.905±0.077, respectively). *p<0.05 refers to corresponding STZ or STZ-IgG animals.

FIG. 4 is graphs of experimental results demonstrating that neutralization of MIF activity reduces the production of TNF-α. Spleen MNC(SMNC), peritoneal cells (PC) and pancreatic islets were isolated from control untreated mice (control), mice treated with STZ and non-immune rabbit IgG (STZ-IgG), STZ and anti-MIF IgG (STZ-αMIF), STZ and vehicle (STZ), and STZ and ISO-1 (STZ-ISO). TNF production was measured in the cell culture supernatants as described in Materials and Methods of the Example. Results are representative of three independent experiments with similar results. *p<0.05 refers to corresponding STZ-IgG (Panel A) or STZ (Panel B) animals.

FIG. 5 is graphs of experimental results demonstrating that neutralization of MIF activity down-regulates the expression of iNOS and NO production. Peritoneal cells (PC) and pancreatic islets were isolated from mice treated as described in FIG. 3. In Panel A, iNOS expression was determined by cell-based ELISA, and presented as fold increase compared to control value (O.D. 492 nm 0.445±0.027). In Panel B, after isolation from mice, PC were cultivated in medium for 48 hours, and pancreatic islets in the presence of 250 U/ml IFN-γ+IL-1β for 72 hours. Subsequently, nitrite accumulation in cell culture supernatants was determined. Results are representative of three independent experiments with similar results. *p<0.05 refers to corresponding STZ or STZ-IgG animals.

FIG. 6 is graphs of experimental results demonstrating that neutralization of MIF activity does not affect the expression of IFN-γ and MHC class II. Spleen MNC(SMNC) and peritoneal cells (PC) were isolated from mice treated as described in FIG. 3. In Panel A, IFN-γ expression in SMNC was determined by cell-based ELISA, and presented as fold increase compared to control value (O.D. 492 nm 0.070±0.014). In Panel B, MHC II molecules expression in PC and SMNC was determined by cell-based ELISA, and presented as fold increase compared to control value (O.D. 492 nm 1.678±0.151 and 1.204±0.124, respectively). Results are representative of three independent experiments with similar results. *p<0.05 refers to corresponding STZ or STZ-IgG animals.

FIG. 7 is a graph of experimental results showing inhibition of diabetes in streptozotocin-treated mice that were treated with an MIF inhibitor.

FIG. 8 is a graph of experimental results showing the effect of timing of MIF inhibitor ISO-1 and anti-MIF antibody on control of diabetes in streptozotocin-treated mice. Blood glucose levels in untreated CBA/H mice (control, n=8), or of 21 day MLD-STZ diabetic mice treated with either STZ alone (STZ, n=8), or STZ and ISO-1 given as an early (STZ-ISO Early, n=8), Late (STZ-ISO-1 Late, n=8) or Late anti-MIF Ab (STZ-•-MIF, n=8). Early injection: ISO-1 was administered by i.p. injection at 40 mg/Kg/day for 14 days and started 3 days prior to STZ first injection; Late injection: ISO-1 or anti-MIF Ab was administered on day six, after the last injection of STZ. *p<0.05 refers to corresponding STZ animals.

FIG. 9 is a graph of experimental results showing that MIF-null mice do not acquire diabetes after treatment with streptozotocin. Blood glucose levels were determined in wild-type and MIF⁺ C57B1/6 mice. Animals received MLD-STZ injections (5×40 mg/kg/day). Blood glucose levels were determined starting at the beginning of the STZ treatment and continued through weekly measurements. *p<0.005 refers to corresponding STZ animals. Blood glucose levels were determined: (a) starting at the beginning of the STZ treatment and continued through weekly measurements. Symbols used for the respective two groups which received MLD-STZ injections: C57B1/6 mice (; n=11) and MIF−/− C57B1/6 (o; n=11). Results from a representative of two independent experiments are presented as ±SD. *p<0.005 refers to corresponding STZ animals. Statistical analysis was performed by ANOVA with Bonferroni's adjustment.

### DETAILED DESCRIPTION OF THE INVENTION

Abbreviations: MIF, macrophage migration inhibitory factor; ISO-1, (S,R)-3-(4-hydroxyphenyl)-4,5-dihydro-5-isoxazole acetic acid methyl ester; iNOS, inducible nitric oxide synthase; STZ, streptozotocin; MLD-STZ, multiple low doses of streptozotocin; SMNC, spleen mononuclear cells; PC, peritoneal cells; IFN-γ, γ-interferon; TNF-α, tumor necrosis factor-α; IL-1β, interleukin-1β.

The present invention is based on the discovery that MIF is a critical factor in the pathogenesis of type 1 diabetes. Therefore, inhibition of MIF prevents or attenuates the development of type 1 diabetes. See Examples.

Thus, in some embodiments, the invention is directed to methods of treating a mammal having type 1 diabetes or at risk for type 1 diabetes. The methods comprise administering to the mammal a pharmaceutical composition comprising an agent that inhibits a macrophage migration inhibitory factor (MIF) in the mammal. In these embodiments, the agent is a polypeptide or a polynucleotide.

In some of these methods, the agent inhibits activity of the MIF. One type of such an agent comprises an antibody binding site that binds specifically to the MIF, for example an antibody, an Fab fragment or an F(ab)₂ fragment of an antibody. Such agents can be produced by well-known methods. Non-limiting methods include: immunization of animals with MIF, followed by isolation of anti-MIF antibodies from serum or production of anti-MIF monoclonal antibodies from hybridomas made by fusion of spenocytes with myeloma cells; or phage display or other recombinant methods. Preferably, the monoclonal antibody is chosen or adapted to match the species to be treated. For treatment of humans, for example, the anti-MIF antibody (or antigen-binding fragment thereof) will be a human antibody or a humanized antibody. Such antigen-specific human or humanized monoclonal antibodies may be developed by a variety of methods well known in the art.

Another agent useful for these methods that inhibits activity of the MIF is an aptamer that binds specifically to the MIF. Aptamers are single stranded oligonucleotides or oligonucleotide analogs that bind to a particular target molecule, such as MIF. Thus, aptamers are the oligonucleotide analogy to antibodies. However, aptamers are smaller than antibodies, generally in the range of 50-100 nt. Their binding is highly dependent on the secondary structure formed by the aptamer oligonucleotide. Both RNA and single stranded DNA (or analog), aptamers are known. See, e.g., 5,773,598; 5,496,938; 5,580,737; 5,654,151; 5,726,017; 5,786,462; 5,503,978; 6,028,186; 6,110,900; 6,124,449; 6,127,119; 6,140,490; 6,147,204; 6,168,778; and 6,171,795. Aptamers can also be expressed from a transfected vector (Joshi et al., 2002, J. Virol. 76,6545).

Aptamers that bind to virtually any particular target can be selected by using an iterative process called SELEX, which stands for Systematic Evolution of Ligands by EXponential enrichment (Burke et al., 1996., J. Mol. Biol. 264, 650; Ellington and Szostak, 1990, Nature 346,818; Schneider et al., 1995, Biochemistry 34, 9599; Tuerk and Gold, 1992, Proc. Natl. Acad. Sci. USA 89:6988; Tuerk and Gold, 1990, Science 249:505). Several variations of SELEX have been developed which improve the process and allow its use under particular circumstances. See, e.g., U.S. Pats. No. 5,472,841; 5,503,978; 5,567,588; 5,582,981; 5,637,459; 5,683,867; 5,705,337; 5,712,375; and 6,083,696. Thus, the production of aptamers to any particular peptide, including MIF, requires no undue experimentation.

In other embodiments of these methods, the agent inhibits expression of the MIF. Preferred examples include inhibitory oligonucleotides in the form of antisense nucleic acids, ribozymes and small inhibitory nucleic acids (e.g., siRNA) specific for MIF mRNA in the mammal. Each of these inhibitory oligonucleotides requires knowledge of the sequence of MIF mRNA. MIF mRNA sequences for many mammalian species are known. See, e.g., SEQ ID NO: 1-3, providing MIF cDNA sequences for human, mouse and rat, respectively. The MIF cDNA sequence for any mammal could be determined without undue experimentation, e.g., by amplifying the sequence from a cDNA preparation of the mammal, using primers designed from a known mammalian MIF cDNA sequence.

In some aspects of these embodiments, the inhibitory oligonucleotides of the present invention are antisense nucleic acids or mimetics. Antisense nucleic acid molecules act to directly block the translation of mRNA by hybridizing to targeted mRNA and preventing protein translation. Antisense approaches involve the design of oligonucleotides that are complementary to a portion of an MIF mRNA. The antisense oligonucleotides will bind to the complementary protective sequence mRNA transcripts and prevent translation. Absolute complementarity, although preferred, is not required.

Ribozyme molecules designed to catalytically cleave MIF mRNA transcripts can also be used to prevent translation of MIF mRNA and, therefore, expression of the MIF protein. See, e.g., PCT Publication WO 90/11364; Sarver, et al., 1990.

Ribozymes are enzymatic RNA molecules capable of catalyzing the specific cleavage of RNA. For a review, see Rossi, 1994. The mechanism of ribozyme action involves sequence specific hybridization of the ribozyme molecule to complementary target RNA, followed by an endonucleolytic cleavage event. The composition of ribozyme molecules must include one or more sequences complementary to the MIF mRNA, and must include the well known catalytic sequence responsible for mRNA cleavage. For this sequence, see, e.g., U.S. Pat. No. 5,093,246.

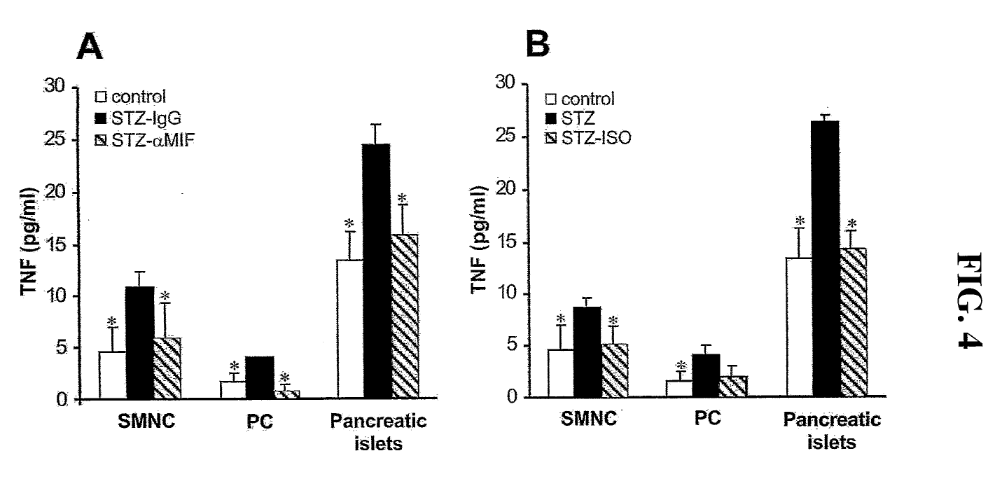

Preferred types of ribozymes for the present invention are hammerhead ribozymes. In these embodiments the hammerhead ribozymes cleave MIF mRNA at locations dictated by flanking regions that form complementary base pairs with the mRNA. The sole requirement of the hammerhead ribozyme is that the mRNA have the two base sequence 5′-UG-3′, which occurs numerous times in the MIF gene (see SEQ ID NO: 1-3). The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988.

The ribozymes of the present invention also include RNA endoribonucleases (hereinafter “Cech-type ribozymes”) such as the one which occurs naturally in Tetrahymena thermophila (known as the IVS, or L-19 IRS RNA) and which has been extensively described by Thomas Cech and collaborators (Been and Cech, 1986; Zaug, et al., 1984; Zaug and Cech, 1986; Zaug, et al., 1986; WO SS/04300, Cell, 47:207-216). The Cech-type ribozymes have an eight base pair active site that hybridizes to the MIF mRNA sequence wherever cleavage of the MIF RNA is desired. The invention encompasses those Cech-type ribozymes that target eight base-pair sequences that are present in the MIF mRNA.

As with the antisense approach, the ribozymes can be composed of modified oligonucleotides (e.g., for improved stability, targeting, etc.) and should be delivered to cells that make MIF in vivo, preferably activated T cells and/or macrophages. A preferred method of delivery involves using a DNA construct “encoding” the ribozyme under the control of a strong constitutive pol III or pol II promoter, so that transfected cells will produce sufficient quantities of the ribozyme to destroy endogenous MIF gene messages and inhibit translation. Because ribozymes, unlike antisense molecules, are catalytic, a lower intracellular concentration is required for efficiency.

Alternatively, endogenous target gene expression can be reduced by targeting deoxyribonucleotide sequences complementary to the regulatory region of the MIF gene (i.e., the MIF gene promoter and/or enhancers) to form triple helical structures that prevent transcription of the MIF gene in target cells in the body. See generally, Helene, 1991; Helene, et al., 1992; Maher, 1992.

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleic acids may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex. Several such GC-rich areas are available for targeting in the MIF gene (SEQ ID NO:1-3).

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

In other embodiments, the oligonucleotide can be a small interfering RNA (siRNA), known in the art to be double stranded RNAs, complementary to the target mRNA (here MIF), that interacts with cellular factors to bind to the target sequence, which is then degraded. The siRNA sequence can be complementary to any portion of the MIF mRNA. The siRNA is preferably 21-23 nt long, although longer sequences will be processed to that length. References include Caplen et al., 2001; Elbashir et al., 2001; Jarvis and Ford, 2002; and Sussman and Peirce, 2002.

Antisense RNA and DNA, ribozyme, triple helix, and siRNA molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid-phase phosphoramidite chemical synthesis. Alternatively, RNA molecules may be generated by in vitro or in vivo transcription of DNA sequences encoding the antisense RNA molecule. Such DNA sequences may be incorporated into a wide variety of vectors that incorporate suitable RNA polymerase promoters such as the T7 or SP6 polymerase promoters. In another alternative, antisense cDNA constructs that synthesize antisense RNA constitutively or inducibly, depending on the promoter used, can be introduced stably into cell lines, or into target cells in the mammal by known gene therapy methods.

As used herein, the term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring portions which function similarly. Such oligonucleotide mimetics are often preferred over native forms because of desirable properties such as, for example, enhanced cellular uptake, enhanced affinity for nucleic acid target and increased stability in the presence of nucleases.

Specific examples of preferred mimetics useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al., Science, 1991, 254, 1497-1500.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂ CH₃, ON₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-OCH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-OCH₂OCH₂N(CH₂)₂, also described in examples hereinbelow.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (CH₂)ₙ group bridging the 2′ oxygen atom and the 3′ or 4′ carbon atom wherein n is 1 or 2. LNAs and preparation thereof are described in WO 98/39352 and WO 99/14226.

Other preferred modifications include 2′-methoxy (2′-OCH₃), 2′-aminopropoxy (2′-OCH₂ CH₂ CH₂ NH₂), 2′-allyl (2′-CH₂CH═CH₂), 2′-O-alkyl (2′-OCH₂CH═CH₂), and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide, Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl, uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine, phenothiazine cytidine, G-clamps such as a substituted phenoxazine cytidine, carbazole cytidine, and pyridoindole cytidine. Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; and 5,750,692, which is commonly owned with the instant application and also herein incorporated by reference.

Another modification of the oligonucleotides of the invention involves chemically linking to the oligonucleotide one or more moieties or conjugates that enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

It is not necessary for all positions in a given compound to be uniformly modified, and in fact more than one of the aforementioned modifications may be incorporated in a single compound or even at a single nucleoside within an oligonucleotide. The present invention also includes inhibitory oligonucleotide compounds that are chimeric compounds. “Chimeric” inhibitory oligonucleotide compounds or “chimeras,” in the context of this invention, are inhibitory oligonucleotides that contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric inhibitory oligonucleotide compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

The inhibitory oligonucleotides useful for the invention methods may be synthesized in vitro. The inhibitory oligonucleotides of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 and U.S. Pat. No. 5,770,713 to Imbach et al.

The oligonucleotides can also comprise a non-nucleotide moiety, such as a hapten, a fluorescent molecule, or a radioactive moiety, useful, e.g., to detect or quantify the amount of oligonucleotide that has entered a cell.

In preferred embodiments, the mammal has or is at risk for having diabetes, impaired glucose intolerance, stress hyperglycemia, metabolic syndrome, and/or insulin resistance.

These methods can be used with any mammal. Preferably, the mammal is a rodent (e.g., a mouse injected with streptozotocin, which is an accepted animal model for type 1 diabetes). In other preferred embodiments, the mammal is a human.

The invention is also directed to methods of treating a mammal having type 1 diabetes or at risk for type 1 diabetes. These methods comprise administering to the mammal a pharmaceutical composition comprising an agent that inhibits a macrophage migration inhibitory factor (MIF) in the mammal. In these embodiments, the agent is an organic molecule comprising the following structure I or II

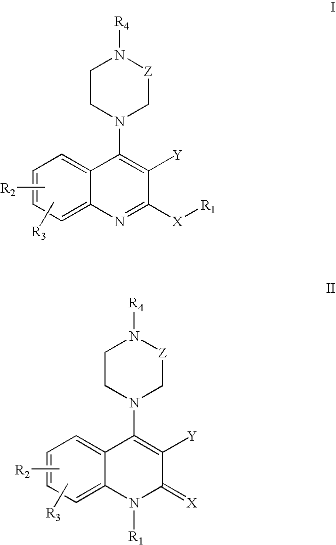

Preferably, the organic molecule comprises structure II, where

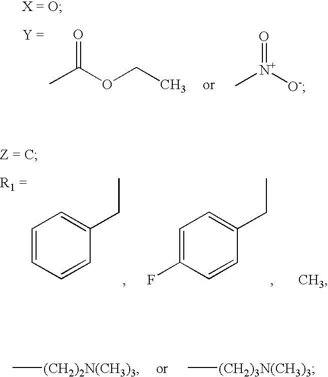

the ring comprising R₂ and R₃=

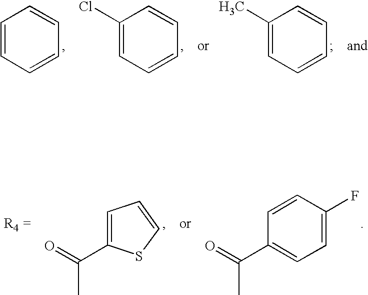

Any of the above-described compositions can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compositions useful for the present invention can easily be administered parenterally such as for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic. Rectal administration includes administering the pharmaceutical compositions into the rectum or large intestine. This can be accomplished using suppositories or enemas. Suppository formulations can easily be made by methods known in the alt. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

In preferred embodiments of the invention, the mammal has or is at risk for having diabetes or conditions associated with diabetes or a risk of diabetes, for example impaired glucose intolerance, stress hyperglycemia, metabolic syndrome, and/or insulin resistance.

The present invention can be utilized for any mammal, for example rodents, generally used as experimental models for diabetes (see Example), or humans.

The discovery that compounds that inhibit MWF activity or expression are useful for treatment of type 1 diabetes indicates that screens of compounds for MIF-inhibiting activity can be used to identify compounds for the ability to prevent or treat type 1 diabetes. Thus, in some embodiments, the invention is directed to methods of evaluating whether a compound is useful for preventing or treating type 1 diabetes. The methods comprise the following two steps: (a) determining whether the compound inhibits a macrophage migration inhibitory factor (MIF) in a mammal, then, if the compound inhibits the MIF, (b) determining whether the compound inhibits development of type 1 diabetes.

In these methods, the ability of the tested compound to inhibit MIF activity can be determined by any known procedure. Non-limiting examples include those methods described in U.S. Patent Application Publications 2003/0008908 and 2003/0195194. Similarly, any known procedure for evaluating the effect of a compound on type 1 diabetes can be utilized. In preferred embodiments, the effect of the compound on type 1 diabetes is determined by evaluating the effect of the compound on development of diabetes in animal models utilizing multiple low dose streptozotocin administration or in animal models genetically susceptible to development of type 1 diabetes, such as the NOD mouse. The effect of a test compound or formulation on the development of diabetes in such models may be assessed by a variety of convenient methods, for instance by monitoring circulating glucose or proliferation of splenic lymphocytes in the mammal (see also Example).

These methods are not limited to evaluation of any particular type of compound. Examples of compounds that can be evaluated by these methods include oligopeptides or proteins such as enzymes or proteins that comprise an antibody binding site; nucleic acids or mimetics such as antisense compounds, ribozymes, aptamers, interfering RNAs such as siRNAs. In preferred embodiments, the compound is an organic molecule less than 1000 Dalton having structure I or structure II, as previously defined.

In additional embodiments, the invention is directed to kits comprising (a) a pharmaceutical composition comprising an agent that inhibits a macrophage migration inhibitory factor (MIF) in the mammal, where the agent is a polypeptide or a polynucleotide, and (b) instructions for administering the composition to the mammal. In these embodiments, the mammal has type 1 diabetes or is at risk for type 1 diabetes.

In further embodiments, the invention is directed to other kits comprising (a) a pharmaceutical composition comprising an agent that inhibits a macrophage migration inhibitory factor (MIF) in the mammal, and (b) instructions for administering the composition to the mammal. In these embodiments, the mammal has type 1 diabetes or is at risk for type 1 diabetes. The pharmaceutical composition of these embodiments is an organic molecule comprising the following structure I or II

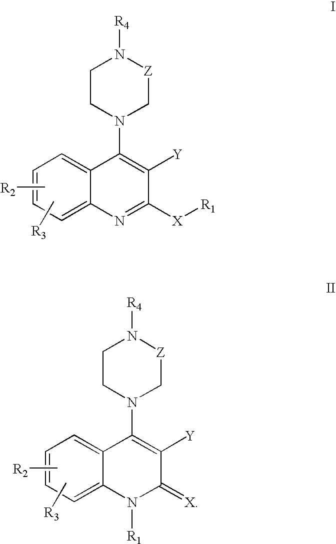

The invention is also directed to the use of an agent that inhibits a macrophage migration inhibitory factor (MIF) in the mammal, where the agent is a polypeptide or a polynucleotide, for the manufacture of a medicament for the treatment of a mammal having type 1 diabetes or at risk for type 1 diabetes.

Additionally, the invention is directed to the use of an agent that inhibits a macrophage migration inhibitory factor (MIF) in the mammal for the manufacture of a medicament for the treatment of a mammal having type 1 diabetes or at risk for type 1 diabetes. In these embodiments, the agent is an organic molecule comprising the following structure I or II

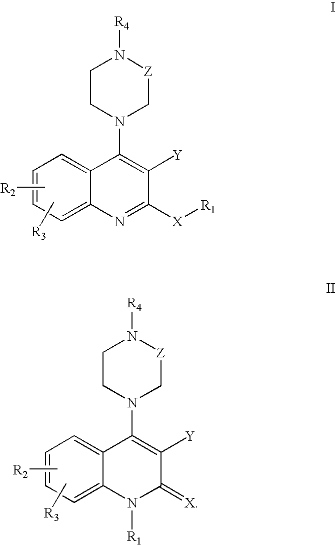

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1

### Neutralization of Macrophage Migration Inhibitory Factor (MIF) Activity Attenuates Experimental Autoimmune Diabetes

### Example Summary

The pro-inflammatory cytokine, macrophage migration inhibitory factor (MIF), plays a pivotal role in several inflammatory and autoimmune diseases. MIF mRNA expression is up-regulated in non-obese diabetic mice, yet little is known about its potential as a regulator of type 1 diabetes. Here, we show that MIF protein is significantly elevated in islet cells during the development of experimental diabetes induced in mice by multiple low doses of streptozotocin. Attenuation of MIF activity with exemplary MIF inhibitors such as neutralizing antibodies against MIF, or the pharmacological MIF inhibitor (S,R)-3-(4-hydroxyphenyl)-4,5-dihydro-5-isoxazole acetic acid methyl ester (ISO-1), markedly reduces histopathological changes in the islets of pancreas and suppresses the development of hyperglycaemia, showing the general utility of this method of treating mammals with, or at risk for, type 1 diabetes. The observed beneficial effects could be attributed to the reduced proliferation and adhesion of autoreactive lymphocytes, down-regulation of iNOS expression, as well as NO and TNF-α secretion by islets of pancreas and by peritoneal macrophages, although knowledge of these mechanistic links are not essential to the teaching of the inventive method. This study defines a critical role for MIF in the pathogenesis of type 1 diabetes and identifies a new therapeutic strategy to attenuate the autoimmune process at multiple levels.

### Introduction

The observation that MIF also stimulates the synthesis of other proinflammatory cytokines and soluble mediators involved in the pathogenesis of type 1 DM such as TNF-(t, IL-1 and nitric oxide (NO) (19) raised the possibility that inhibition of MIF activity may modulate the development of disease.

Given the central regulatory role of MIF in the immune response mediated by both macrophages and T cells, we have investigated the expression of MIF during the progress of diabetes in mice treated with multiple low doses of streptozotocin (MLD-STZ), as well as the influence of MWF activity neutralization on the disease development. These are well-accepted models for type 1 diabetes in humans. For this purpose, an anti-MIF polyclonal antibody, as well as (S,R)-3-(4-hydroxyphenyl)-4,5-dihydro-5-isoxazole acetic acid methyl ester (ISO-1), a pharmacological inhibitor of MIF (20), were given to mice as prophylactic treatment prior to the challenge with STZ. Various immunological parameters relevant for DM pathogenesis were determined, including autoreactive cell proliferation and adhesion, as well as the production of proinflammatory mediators. To our knowledge, this is the first attempt to interfere in autoimmune diabetes by in vivo neutralization of MIF.

### Materials and Methods

Mice. Inbred CBA/H mice were obtained from our own breeding colony at the Institute for Biological Research, Belgrade. Inbred C57 B1/6 mice were originally purchased from Charles River (Calco, Italy) and then bred by brother per sister mating for up to 4 generations. Mice were kept under standard laboratory conditions with free access to food and water. The handling of the mice and the study protocol were approved by the local Institutional Animal Care and Use Committee.

Reagents. Streptozotocin (STZ, S-0130), sulfanilamide, naphthylenediamine dichidrochloride and irrelevant rabbit IgG were purchased from Sigma (St. Louis, Mo.). Anti-murine MIF IgG was prepared from rabbit serum raised against murine MIF and purified by protein A affinity chromatography following the manufacturer's instructions (Pierce, Rockford, Ill.). ISO-1, [(S,R)-3-(4-hydroxyphenil)-4,5-dihydro-5-isoxasole acetic acid methyl ester] was synthesized as previously described (20).

Diabetes induction and in vivo treatments. Diabetes was induced in adult male mice with multiple subtoxic doses of streptozotocin (MLD-STZ, 40 mg/kg body wt/day i.p. for five consecutive days) as described (21). The impact of polyclonal antibody against MIF was studied by i.p. injection of mice (5-10 per treatment group) with 5 mg/kg of rabbit IgG antibody against mouse MWF on days −3, −1, 2 and 5 in relation to the first STZ dose. The effect of ISO-1 was studied by i.p. injection of the drug at a dose of 1 mg/mouse/day, for 14 consecutive days, starting 3 days prior to the first injection with STZ. Control animals received either nonimmune IgG or ISO-1 diluent (DMSO/H₂O). Mice were monitored for diabetes by weekly measurement of blood glucose levels using a glucometer (Sensimac®, Imaco, Ludersdorf, Germany). Clinical diabetes was defined by hyperglycemia in non-fasted animals (blood glucose over 11.8 mmol/l).

Cell incubation and determination of pro-inflamatory mediators. Resident peritoneal cells (PC), spleen mononuclear cells (SMNC), and pancreatic islets were isolated from individual anti-MIF IgG-treated, ISO-1-treated or control diabetic mice on day 15 after the first injection of STZ, as well as from normal untreated animals. SMNC (5×10⁶/well) prepared by Ficoll gradient centrifugation, resident PC (2.5×10⁵/well) collected by peritoneal lavage with cold PBS, or pancreatic islets (150-200 islets/well) prepared by collagenase digestion and density gradient purification as described (22), were incubated in 24-well Limbro culture plates in 1 ml of RPMI-1640 culture medium containing 5% fetal bovine serum (FBS) and cell supernatants were collected after 48 h. Concentration of bioactive TNF-α in culture supernatant was determined as previously described (22) using a cytolytic bioassay with actinomycin D-treated fibrosarcoma cell line L929. Nitrite accumulation, an indicator of NO production, was determined in cell culture supernatant using the Griess reaction (22). The expression of intracellular MIF, IFN-γ, MHC II, or iNOS was determined by slight modification of cell-based ELISA protocol (23). Spleen MNC (5×10⁵/well) or PC (2.5×10⁵/well), were allowed to adhere to poly-L-lysine-precoated 96-well microplates. Following fixation with 4% paraformaldehyde, and washing with 0.1% Triton X-100 in PBS (T/PBS), endogenous peroxidase was quenched with 1% H₂O₂ in T/PBS, and reaction blocked for 1 h at 37° C. with 10% FBS in T/PBS. Subsequently, cells were incubated for 1 h at 37° C. with either rabbit anti-mouse MIF IgG, rat anti-mouse IFN-γ (Holland Biotechnology, Leiden, The Netherlands), mouse anti-mouse/rat MHC II (MCA46R, Serotec), or rabbit anti-mouse iNOS (Sigma), in T/PBS containing 1% BSA. After washing, the cells were incubated for 1 h with the corresponding secondary antibody (goat anti-rabbit Ig(H+L)-HRP, goat anti-rat Ig(H+L)-HRP or goat anti-mouse Ig(F(ab′)₂-specific)-HRP washed again and incubated for 15 min at room temperature in dark with 50 μl of a solution containing 0.4 mg/ml OPD (Sigma), 11.8 mg/ml Na₂HPO₄×2H₂O, 7.3 mg/ml citric acid and 0.015% H₂O₂. The reaction was stopped with 3N HCl, and the absorbance was measured in a microplate reader at 492 nm in a Titertek microplate reader (Flow).

Ex vivo lymphoproliferative response and adhesion assay. Spontaneous proliferation of SMNC was determined by incubation of cells (5×10⁵/well) from each individual animal in 96-well microplates with 1 μCi ³H-thymidine (³H-TdR, ICN). Incorporated radioactivity was measured after 24 h in a Beckman liquid scintillation counter. The analysis of spontaneous adhesion of SMNC (2.5×10⁵/well) to a monolayer of L929 fibroblasts or plastic was performed by using crystal violet assay as previously described (21). The absorbance corresponding to the number of adherent cells, was measured at 570 nm.

Antibodies and flow cytometry. Spleen MNC (1×10⁶) were incubated with the rat anti-mouse monoclonal antibodies (mAbs): anti-CD11b (MAC-1)-phycoerythrin (PE), or anti-CD25 (IL-2 Receptor α chain, p55)-biotin (PharMingen, San Diego, Calif.), followed by Streptavidin-PE (PharMingen). Each cell suspension of SMNC was a pool from three to five animals obtained from the same experimental group 15 days after diabetes induction, as well as from normal untreated mice. Cell surface marker expression was analyzed with a flow cytometer (FACScalibur, Becton Dickinson, Heidelberg, Germany) and Win MDI 2.8 software (Joseph Trotter).

Histological and immunohistochemical examination of pancreas. Pancreata were fixed in neutral buffered formalin and then embedded in paraffin. The fixed blocks were sectioned (7 μm thick) and haematoxylin and eosin staining was performed to assess the incidence and degree of inflammatory changes. Insulitis scoring was performed as previously described (10, 11) by examining at least 15 islets per mouse and graded in a blinded fashion as follows: 0, no infiltrate; 1, peri-ductal infiltrate; 2, peri-islet infiltrate; 3, intraislet infiltrate; and 4, intraislet infiltrate associated with β-cell destruction. At least 15 islets were counted for each mouse. A mean score for each pancreas was calculated by dividing the total score by the number of islets examined. Insulitis scores (IS) are expressed as mean values±SD. Immunohistochemistry was performed on paraffin-embedded sections of formalin-fixed tissue using a previously described microwave-based method (24). For MIF immunostaining, a polyclonal rabbit anti-MIF IgG and control rabbit IgG were used. The examined area of the islet was outlined, and the percentage of MIF-positive islet cells was measured using quantitative Image Analysis System (Optima 6.5, Media Cybernatics, Silver Springs, Md.).

Statistical analysis. The blood glucose values are shown as mean values±SE. Statistical analyses were performed by ANOVA with Bonferronii's adjustment and Fisher's exact test. The other values were expressed as means±SD and groups of data were compared using Student's paired t-test. The results of a representative of at least three separate experiments with similar results are presented. Statistical significance was set at P<0.05.

### Results

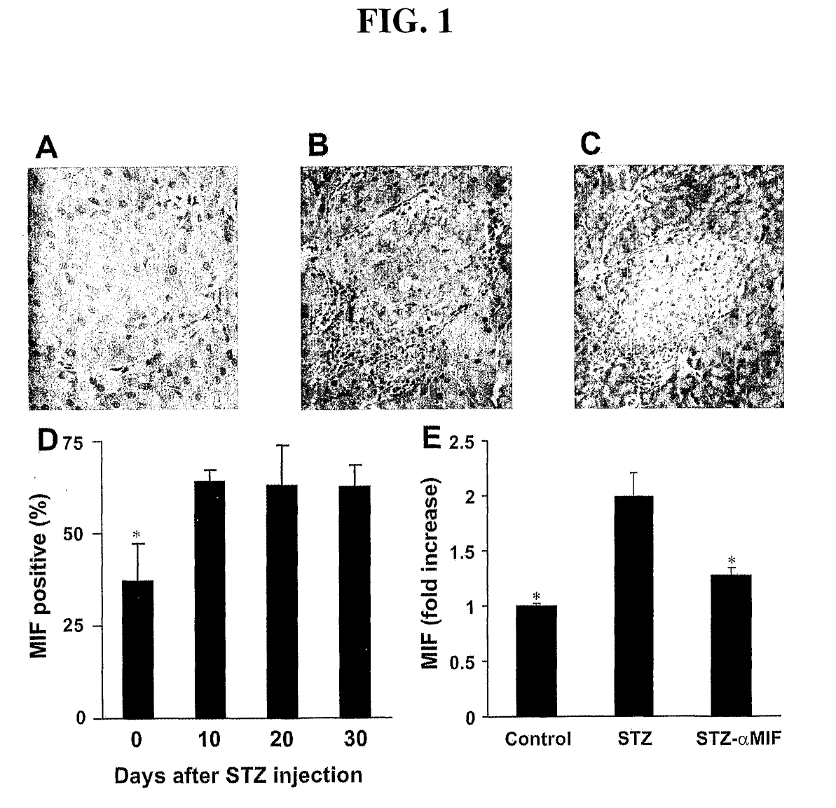

Increased MIF expression in MLD-STZ-induced diabetic mice. Recent studies have revealed that MIF mRNA expression is up-regulated in spontaneously diabetic NOD mice, but its functional role in disease progression is unknown. In order to determine if MIF protein expression levels are altered during immunoinflammatory diabetes, MLD-STZ was administered to diabetes-susceptible CBA/H mice. Immunohistochemistry revealed substantial induction of MIF protein expression by the islet cells in pancreas sections from these mice during the disease course, as well as mononuclear cell infiltration (FIG. 1B-D). Likewise, higher concentrations of MIF were detected in the peritoneal cells (PC) of diabetic mice, in comparison to non-diabetic, control mice (FIG. 1E). In vivo treatment of mice with neutralizing anti-MIF immunoglobulin during disease induction significantly suppressed PC expression of MIF (FIG. 1E). Thus, as a consequence of DM induction, increased MIF-protein content was observed both at the level of peripheral and target tissue, and MIF protein levels can be attenuated by administration of anti-MIF antibodies.

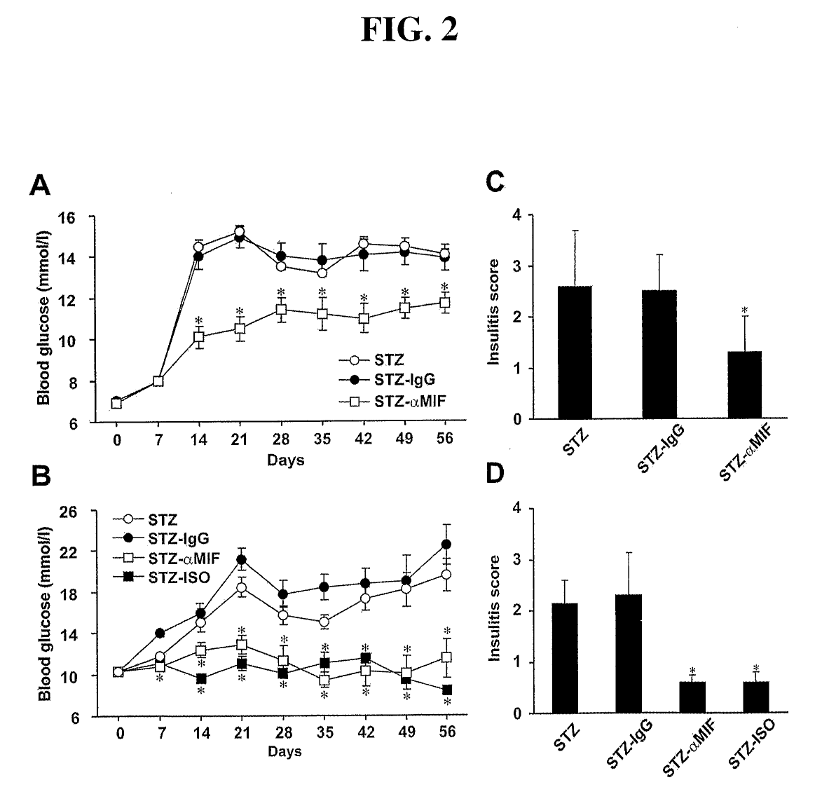

Anti-MIF prophylaxis suppresses clinical and histological parameters of MLD-STZ-induced diabetes. To determine whether inhibition of MIF activity modulates disease in MLD-STZ-exposed mice, we first studied the effect of a neutralizing polyclonal antibody against MIF in two DM-susceptible inbred mouse strains. Both C57B1/6 and CBA/H control mice treated with PBS or non-immune IgG developed sustained hyperglycemia over a 2-week period following MLD-STZ injections. Although MLD-STZ induced different degrees of hyperglycemia in the two mouse strains, treatment of either C57B1/6 mice (FIG. 2A), or CBA/H mice (FIG. 2B) with anti-MIF Ab from day −3 to day +5 significantly inhibited MLD-STZ-induced hyperglycemia. In addition, histological examination of pancreatic specimens performed on day 15 of the disease indicated that the degree of islet infiltration was significantly lower in mice treated with anti-MIF antibodies as compared with diabetic control mice treated with irrelevant IgG (FIGS. 2C and D). We also examined the effect of the pharmacological MIF antagonist ISO-1 (20) in MLD-STZ-induced DM. Accordingly, we treated mice prophylactically over a period of 14 days with ISO-1, starting 3 days before the first of five injections of STZ. These mice remained euglycemic throughout the eight-week experimental period (FIG. 2B). The anti-diabetogenic effect of either immunological or pharmacological neutralization of MIF was long-lasting, with limited variations throughout the entire 56 day follow-up period (FIGS. 2A and B). MIF blockade by ISO-1 attenuated inflammation of the islets (FIG. 2D). These data provide evidence for a critical role of MIF in the induction and progression of immunoinflammatory DM and for the utility of treatment with MIF inhibitors to avert the development of type 1 diabetes in at-risk populations.

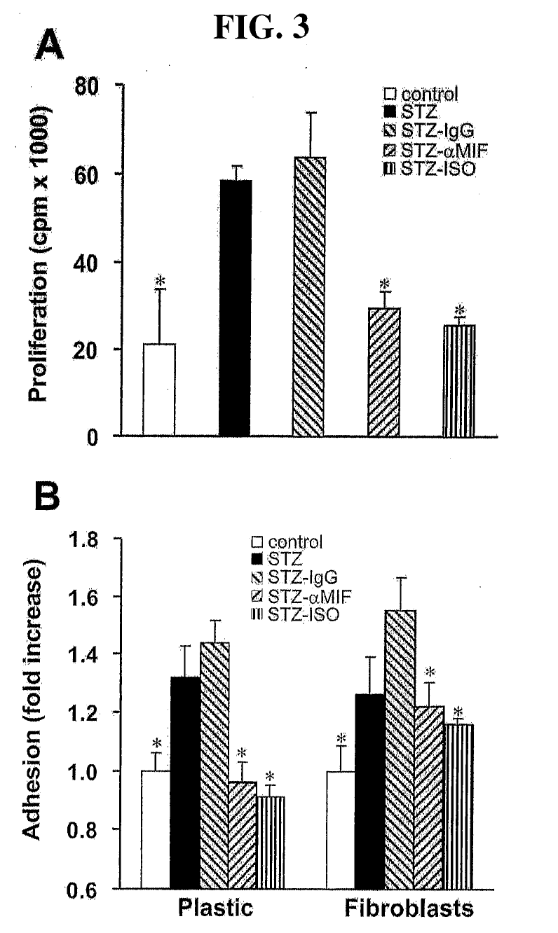

Anti-MIF treatments reduce splenocyte proliferation and adhesive properties. To understand the cellular effects of anti-MIF treatments, ex vivo analysis of the functional characteristics of spleen mononuclear cells (SMNC) from CBA/H mice was performed during early progression of hyperglycemia, on day 15 of hyperglycemia. Previous studies have shown that isolated autoreactive lymphocytes from diabetic mice exhibit significantly increased ex vivo spontaneous proliferation in comparison to cells isolated from healthy animals, suggesting that this cell type may contribute to disease progression (21, 22). To determine if MIF inhibitors modulate splenocyte proliferative responses ex vivo, diabetic CBA/H mice were treated with either anti-MIF antibodies or ISO-1, euthanized at day 15 after MLD-STZ exposure, and splenocytes harvested for ex vivo analyses. Treatment of MLD-STZ-exposed mice with anti-MIF antibodies significantly inhibited diabetes-associated SMNC proliferation (3H-thymidine incorporation was 29782±3694 cpm versus 63651±10157 cpm of diabetic control cells) (FIG. 3A). Likewise, in vivo inhibition of MIF activity with ISO-1 reverted splenocyte proliferation to near-control levels (25637±2018 cpm in comparison to 21150±12723 cpm of untreated control cells) (FIG. 3A). In addition to cell proliferation, cell-cell adhesion, mediated by the interaction between CD11b and ICAM-1 (CD54), participates in the immunological processes of type 1 DM (21, 25), To determine possible consequences of anti-MIF treatment on the interactions between cells participating in the diabetogenic processes, we assessed the adhesive properties of SMNC and found that spontaneous adhesion of cells to plastic, as well as adhesion to fibroblasts was significantly inhibited by in vivo treatment with anti-MIF IgG, or ISO-1 (FIG. 3B). Down-regulation of adhesiveness was associated with the changes of CD11b expression, as revealed by flow cytometry analysis, using antibodies directed against CD11b, a molecule that mediates cellular adhesion to ICAM-1 (CD54). Thus, in comparison to SMNC derived from control diabetic mice, both anti-MIF IgG treatment and ISO-1 treatment (Table 1) reduced the frequency of CD11b⁺ SMNC and mean antigen density (mean fluorescence intensity, MFI) to the level of untreated normal mice, indicative of the anti-diabetogenic benefits of treatment with the MIF inhibitors.

TABLE 1
MIF antagonists down-regulate the expression of CD11b and
CD25 of splenic mononuclear cells

| | |
|---|---|
| CD11b⁺ | CD25⁺ |

| Treatment groups | %* | MFI** | % | MFI |
|---|---|---|---|---|
| Exp. 1 | | | | |
| Untreated | 7.9 | 44.4 | 4.4 | 57.8 |
| STZ + IgG | 9.4 | 72.0 | 8.5 | 77.7 |
| STZ + αMIF | 6.4 | 48.6 | 4.3 | 70.6 |
| Exp. 2 | | | | |
| Untreated | 15.5 | 88.1 | 2.5 | 96.2 |
| STZ | 19.4 | 108.3 | 6.1 | 113.0 |
| STZ + ISO-1 | 15.3 | 88.5 | 4.5 | 106.0 |

Data of two out of five experiments with similar results obtained by flow cytometry analysis from the pull of three mouse spleens per group.
*Frequency of positive cells.
**Mean fluorescence intensity.

Since IL-2 is centrally involved in the initiation of any immune response, negating its action by blocking the interaction with the IL-2 receptor (IL-2R, CD25) system has gained much attention as a possible therapeutic target for immunointervention in both rodent and human disease (26). Anti-CD25 has also been shown to attenuate low-dose streptozotocin-induced diabetes in mice (12, 26). To determine the effect of in vivo treatment with MWF antagonists on W-2R⁺ cell populations, lymphocytes were isolated from the spleens of control or anti-MIF-treated mice 15 days post-MLD-STZ exposure and labeled with fluorescently tagged antibodies directed against IL-2R (CD25). Staining the cells for IL-2R expression (Table 1) revealed significant increase in both the percentage of IL-2R⁺ lymphocytes and antigen density in control diabetic mice, when comparing to normal untreated animals, while the in vivo treatment with anti-MIF IgG, or with ISO-1 reduced these parameters.

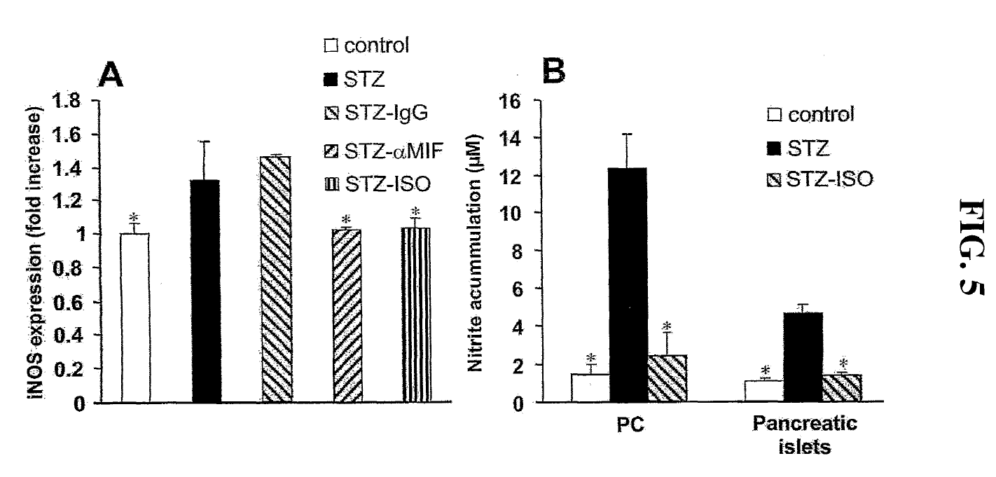

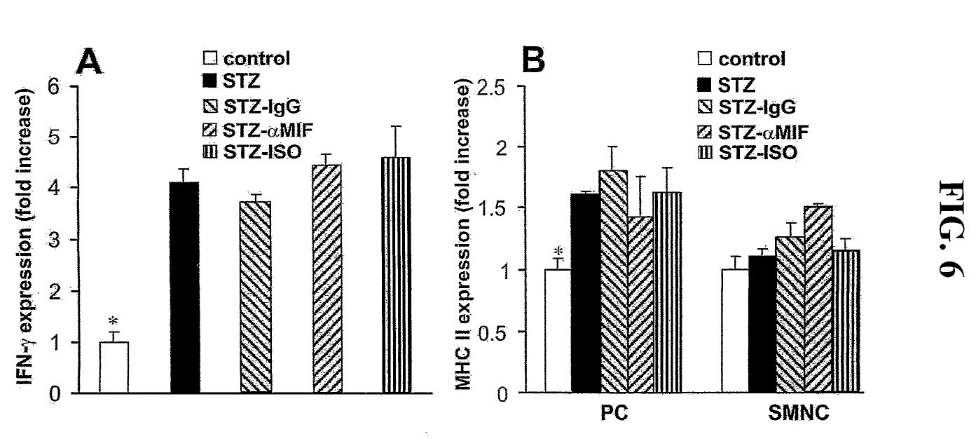

Production of pro-inflammatory mediators. Since proinflammatory mediators play crucial role in type 1 DM development in rodents (4-7, 21, 22), we determined the effects of MIF inhibition on STZ-associated cytokine release from both local and peripheral immune cells ex vivo. Immunological neutralization of endogenous MIF protein with anti-MIF IgG (FIG. 4A), or pharmacological inhibition of MIF with ISO-1 (FIG. 4B), down-regulated TNF-α production by spleen MNC, peritoneal macrophages and pancreatic islet cells, reducing it to the level of healthy, nontreated mice. We also found that intracellular expression of iNOS was significantly reduced in macrophages isolated from animals treated with either anti-MIF or ISO-1 in comparison to relatively high iNOS expression of diabetic animals (FIG. 5A). Accordingly, MIF blockade abolished subsequent NO production in macrophages, as well as in pancreatic islets (FIG. 5B). The effects of the MIF inhibitors were specific for TNF-α, iNOS, and NO, because the expression of intracellular IFN-γ in the splenocytes, as well as the expression of MHC class II molecules, remained unchanged in comparison to control diabetic mice (FIGS. 6A and B).

### Discussion

MIF significantly contributes to the immunopathology associated with excessive inflammation and autoimmunity, and neutralizing endogenous MIF with neutralizing anti-MIF antibodies, or targeted disruption of the MIF gene, inhibits the development of several rodent models of inflammatory disease, including immunologically induced kidney disease (27), leishmaniasis (28), Gram-negative and Gram-positive sepsis (15, 29, 30), antigen-induced arthritis (31), colitis (16), and experimental autoimmune encephalomyelitis (EAE) (17). Recently, a potential role for MIF in the development and pathogenesis of autoimmune-mediated DM has been implicated in spontaneously diabetic NOD mice, because expression of MIF mRNA is significantly increased during disease development, and exogenous MIF administration increases disease incidence in these animals (18). MIF is constitutively expressed and secreted together with insulin from pancreatic β-cells, and acts as an autocrine factor to stimulate insulin release (32). Because induction of insulin secretion is thought to contribute to immunoinflammatory diabetogenic pathways by favoring the expression on the β-cells and the presentation to the immune cells of antigens that are up-regulated when the functional activity is augmented (12), this “hormonal” property could represent an additional important factor involving endogenous MIF in the initial events of β-cell dysfunction and destruction. Targeting endogenous MIF may therefore be a suitable approach for unraveling the role of this cytokine in the pathogenesis of type 1 DM and for therapeutic and/or prophylactic treatment of the condition.

In the present study, we show for the first time that endogenous MIF plays a key role in the development of murine autoimmune diabetes. Progression of MLD-STZ-induced diabetes was accompanied by up-regulated MIF protein expression both in pancreatic islets and peripheral cells. Immunoneutralization of MIF by anti-MIF IgG, or pharmacological inhibition of MIF activity with ISO-1, markedly attenuated the clinical and histological manifestations of the disease. It inhibited the inflammatory responses as well as splenocyte proliferation and adherence ex vivo. The anti-diabetogenic effect of both agents was long-lasting, because mice remained euglycemic during the 8-week follow-up period. Inhibition of autoreactive T cell proliferation, leukocyte adhesion and migration into target tissue, and macrophage activation by these anti-MIF strategies suggest that immunological or pharmacological neutralization of MIF activity may attenuate pathologic autoimmune responses in vivo.

Consistent with the role of MIF in the T-cell activation and mitogenesis (30, 33), we found that MIF blockade considerably decreased IL-2R expression and spontaneous proliferation of splenic lymphocytes measured ex vivo, which most probably reflected the autoreactive T cell response to blood-born pancreatic antigens released upon STZ destruction of β-cells (21, 22). The results therefore indicate that MIF contributes to the clonal size of autoreactive T cells, and that blocking the expansion of diabetogenic T clones might be partly responsible for the protective effect of these anti-MIF strategies.

In addition to clonal expansion of autoreactive cells, the development of the disease is determined by cell-cell interactions mediated by adhesion receptors and ligands both in the induction and the efferent phase of the autoimmune response. In this context, we have shown that mononuclear cells from MLD-STZ-induced diabetic mice are highly adherent to cultured vascular endothelial cells (21), or fibroblasts (FIG. 3B) at a time of peak disease activity; these adhesive properties may be an important mechanism for inflammatory infiltration of target tissue (21). Several lines of evidence demonstrate that up-regulation of CD11b, which mediates cellular adhesion to ICAM-1 (CD54) (34), might have functional consequences on the interactions between cells participating in the immunological processes of type 1 DM (21, 25). MIF blockade significantly inhibited the expression of CD11b on mononuclear cells of diabetic mice and markedly down-regulated leukocyte adhesive properties, suggesting that anti-MIF strategies may impair homing of these cells to the islets of pancreas, thus contributing to the therapeutic benefit of MIF blockade. In favor of this hypothesis, histological analysis showed reduced insulitis in anti-MIF treated mice. Inhibition of MIF activity also has been shown to down-regulate adhesion molecule-dependent target tissue pathology in EAE (17).

Another important finding of this study is that anti-MIF treatment affects the production of pro-inflammatory and cytotoxic mediators. The lower local production of TNF-α and NO may result from the reduction of inflammatory cell influx into pancreas. Based on our ex vivo findings, however, it seems that MIF blockade might also directly influence macrophage and T cell effector function, as suggested by down-regulation of TNF-α, iNOS and NO by peritoneal and/or spleen cells. This is consistent with the in vitro ability of anti-MIF antibodies to interfere with production of these mediators (28). MIF up-regulates in vitro production of TNF-α, as well as of reactive oxygen species and nitrogen metabolites (35). It therefore is conceivable that direct blockade of MIF-mediated iNOS expression and TNF-α synthesis may contribute to down-regulation of tissue-damaging pro-inflammatory mediators in type 1 DM. Thus, we hypothesize that the efficacy of MIF blockade in the treatment of MLD-STZ DM is due to inhibitory effects on the autoimmune/inflammatory response of T cells and macrophages, as well as islet cells.

Interestingly, the effect of systemic blockade of MIF specifically inhibited TNF-α and iNOS-mediated NO production in both local and peripheral cells, because these therapeutic approaches did not diminish peripheral production of the signature Th1 cytokine, IFN-γ. Consistent with the role for IFN-γ in the regulation of MHC class II, the expression of antigens by peritoneal and spleen cells was not impaired after anti-MIF treatments. Although these observations appear to contradict previous findings demonstrating a central role for IFN-γ in diabetes pathogenesis (36), it must be noted that peripheral cytokine production may not mirror the role of cytokines in the development of the organ-specific autoimmune disease. For example we have previously shown in the DP-BB rat model that in an apparently paradoxical fashion, both anti-IFN-γ antibody and exogenously-administered IFN-γ prevented development of autoimmune diabetes (10, 37). As recently reported for EAE, anti-MIF antibody treatment did not affect antigen-specific splenocyte production of IFN-γ, but did reduce IFN-γ-release from antigen-reactive Th1 cells in the target tissue (17). Therefore, it is possible that anti-MIF treatment in our model reduced cytokine production within the microenvironment of the pancreas, but this effect may not have been reflected in our ex vivo analyses. In support of this, we have previously shown a similar modulatory pattern of IFN-γ expression and distribution of MHC class II+ cells in MLD-STZ-induced type 1 DM by different systemically applied pharmacological agents (21, 22). It is known that IFN-γ may possess dicothomic effects on inflammation, exerting proinflammatory effects when produced at the level of the organ targeted by the immune response (e.g. the islet microenvironment), while activating corticosteroid-dependent and independent anti-inflammatory pathways at the systemic level (8). Having in mind the profound ability of MIF to counteract the immunosuppressive effects of glucocorticoids (38), neutralization of MIF activity accompanied with sustained circulating levels of IFN-γ could potentiate systemic anti-inflammatory effects in the treated animals.

MIF is a critical mediator of inflammatory and autoimmune diseases, because neutralizing endogenous MIF activity with anti-MIF antibodies has been effective in animal models of septic shock, colitis, encephalomyelitis, and leishmania infection (15-17, 28), and may be a promising approach for therapy of various human diseases. However, treatment approaches that rely on exogenously administered proteins, including humanized antibodies, may face several challenges, including potential immunogenicity, that suggest the desirability of more preferred embodiments. Anti-cytokine antibodies can form small inflammatory to complexes with cytokines, and thereby exacerbate inflammatory responses (39). For these reasons, alternatives to the therapeutic use of anti-MIF Ig preparations should also be considered for pharmaceutical development. Thus, pharmacological intervention with ISO-1, as a small drug-like molecule that inhibits MIF tautomerase and biological activity (20), may be a more preferred approach for the treatment of MIF-related diseases. Future studies will address the potential use of MIF inhibitors as a treatment, rather than a prophylactic, for regulation of diabetes pathogenesis and presentation.

### EXAMPLE 2

### Delayed MIF Inhibition after Induction of Diabetes

Using methods as described in Example 1, diabetes was induced in adult male mice with multiple subtoxic doses of streptozotocin (MLD-STZ, 40 mg/kg body wt/day i.p. for five consecutive days) as described (21). The effect of MIF inhibition after induction of diabetes was studied by i.p. injection of (S,R)-3-(4-hydroxyphenyl)-4,5-dihydro-5-isoxazole acetic acid methyl ester (ISO-1), a pharmacological inhibitor of MWF (20) at a dose of 1 mg/mouse/day, for 14 consecutive days starting at day 7 after initial streptozotocin administration. Peripheral blood glucose was monitored weekly in these mice and in control mice that were treated identically, except where the ISO-1 treatment was replaced by ISO-1 diluent.

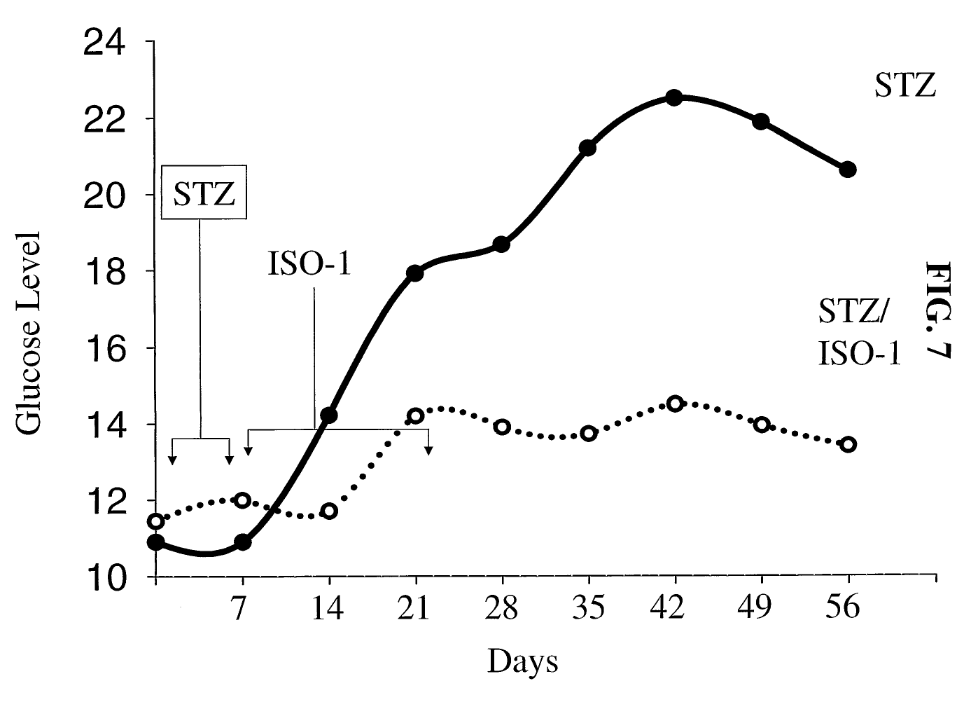

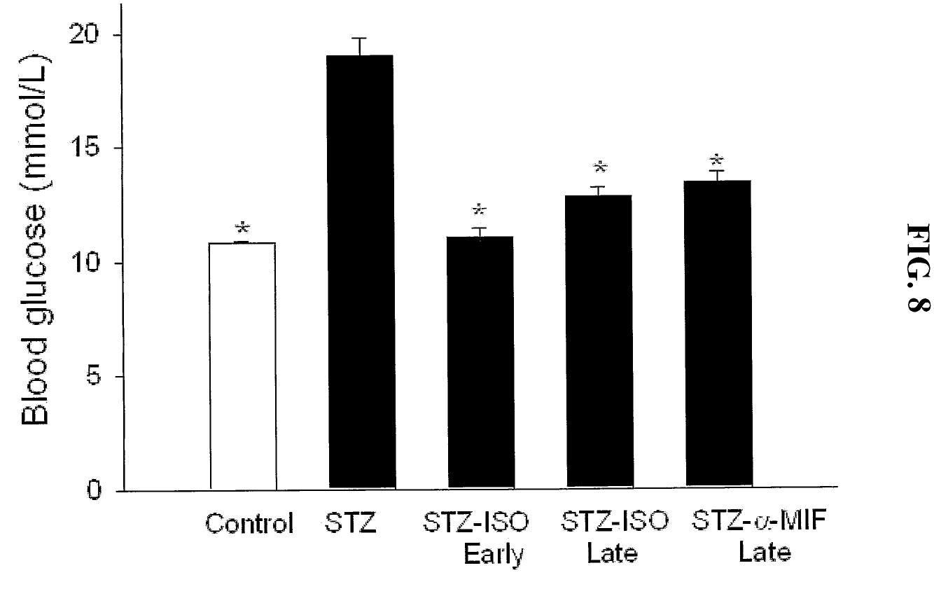

As shown in FIGS. 7 and 8, inhibition of MIF after induction of diabetes provided considerable benefit to the streptozotocin-treated mice, by inhibiting the progression of diabetes.

### EXAMPLE 3

### MIF-null Mice are Resistant to the Induction of Type 1 Diabetes by Streptozotocin

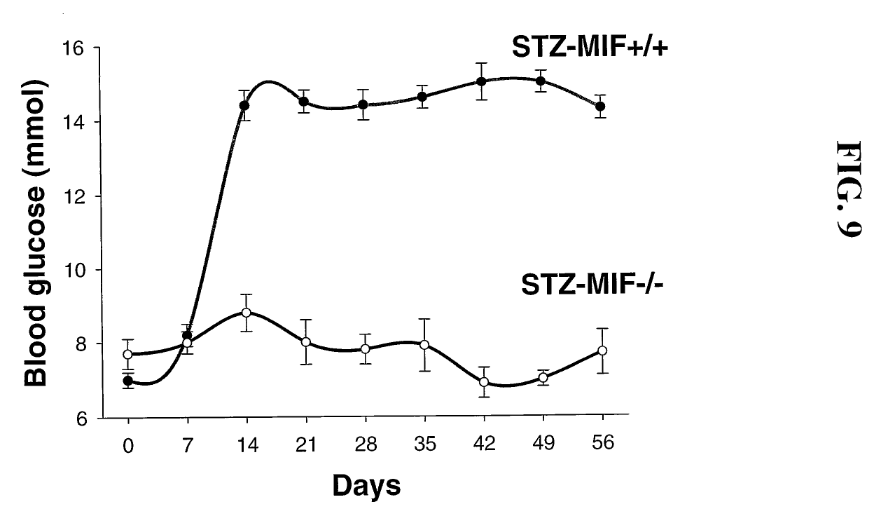

To further establish the importance of MIF in type 1 diabetes, MIF−/− C57B1/6 mice (40) were treated with streptozotocin and compared with MIF^{+/+} mice in progression of diabetes. When treated with streptozotocin, the MIF−/− mice did not develop diabetes, while the MIF+/+ mice did develop diabetes (FIG. 9). This also further establishes that methods directed to inhibiting expression of MIF, e.g., with ribozymes, antisense, siRNA, etc. would be expected to be useful for type 1 diabetes treatment.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

SEQ ID Nos
SEQ ID NO:1 - Human MIF cDNA - GenBank Accession No. NM002415

| | |
|---|---|
| 1 | accacagtgg tgtccgagaa gtcaggcacg tagctcagcg gcggccgcgg cgcgtgcgtc |
| 61 | tgtgcctctg cgcgggtctc ctggtccttc tgccatcatg ccgatgttca tcgtaaacac |
| 121 | caacgtgccc cgcgcctccg tgccggacgg gttcctctcc gagctcaccc agcagctggc |
| 181 | gcaggccacc ggcaagcccc cccagtacat cgcggtgcac gtggtcccgg accagctcat |
| 241 | ggccttcggc ggctccagcg agccgtgcgc gctctgcagc ctgcacagca tcggcaagat |
| 301 | cggcggcgcg cagaaccgct cctacagcaa gctgctgtgc ggcctgctgg ccgagcgcct |
| 361 | gcgcatcagc ccggacaggg tctacatcaa ctattacgac atgaacgcgg ccaatgtggg |
| 421 | ctggaacaac tccaccttcg cctaagagcc gcagggaccc acgctgtctg cgctggctcc |
| 481 | acccgggaac ccgccgcacg ctgtgttcta ggcccgccca ccccaacctt ctggtgggga |
| 541 | gaaataaacg gtttagagac t |

SEQ ID NO:2 - Mouse MIF cDNA - GenBank Accession No. BC024895

| | |
|---|---|
| 1 | ggcttgggtc acaccgcgct ttgtaccgtc ctccggtcca cgctcgcagt ctctccgcca |
| 61 | ccatgcctat gttcatcgtg aacaccaatg ttccccgcgc ctccgtgcca gaggggtttc |
| 121 | tgtcggagct cacccagcag ctggcgcagg ccaccggcaa gcccgcacag tacatcgcag |
| 181 | tgcacgtggt cccggaccag ctcatgactt ttagcggcac gaacgatccc tgcgccctct |
| 241 | gcagcctgca cagcatcggc aagatcggtg gtgcccagaa ccgcaactac agtaagctgc |
| 301 | tgtgtggcct gctgtccgat cgcctgcaca tcagcccgga ccgggtctac atcaactatt |
| 361 | acgacatgaa cgctgccaac gtgggctgga acggttccac cttcgcttga gtcctggccc |
| 421 | cacttacctg caccgctgtt ctttgagcct cgctccacgt agtgttctgt gtttatccac |
| 481 | cggtagcgat gcccaccttc cagccgggag aaataaatgg tttataagag aaaaaaaaaa |
| 541 | aaaaaaa |

SEQ ID NO:3 - Rat MIF cDNA - GenBank Accession No. NM031051

| | |
|---|---|
| 1 | gggtcacgta gtcaggtccc agacttgggt cacaccgcac ttaacaccgt cctccggccg |
| 61 | tcgttcgcag tctctccgcc accatgccta tgttcatcgt gaacaccaat gttccccgcg |
| 121 | cctccgtgcc agaggggttt ctctccgagc tcacccagca gctggcgcag gccaccggca |
| 181 | agccggcaca gtacatcgca gtgcacgtgg tcccggacca gctcatgact tttagtggca |
| 241 | cgagcgaccc ctgcgccctc tgcagcctgc acagcatcgg caagatcggt ggcgcccaga |
| 301 | accgcaacta cagcaagctg ctgtgcggcc tgctgtccga tcgcctgcac atcagcccgg |
| 361 | accgggtcta catcaactat tacgacatga acgcagccaa cgtgggctgg aacggttcca |
| 421 | ccttcgcttg agcccgggcc tcacttacct gcaccgctgt tcttcgagtc ttgctgcacg |
| 481 | ccccgttctg tgtttatcca cccgtaatga tggccacctt ccggtcggga gaaataaatg |
| 541 | gtttgagacc a |

1: A method of treating a mammal having type 1 diabetes or at risk for type 1 diabetes, the method comprising administering to the mammal a pharmaceutical composition comprising an agent that inhibits a macrophage migration inhibitory factor (MIF) in the mammal, wherein the agent is a polypeptide or a polynucleotide.

2: The method of claim 1, wherein the agent comprises a binding site of an antibody that binds specifically to the MIF.

3: The method of claim 2, wherein the agent is an antibody.

4: The method of claim 1, wherein the agent is an aptamer that binds specifically to the MIF.

5: The method of claim 1, wherein the agent inhibits expression of the MIF.

6: The method of claim 5, wherein the agent is an antisense nucleic acid or mimetic specific for MIF mRNA in the mammal.

7: The method of claim 5, wherein the agent is a ribozyme nucleic acid or mimetic specific for MIF mRNA in the mammal.

8: The method of claim 5, wherein the agent is an inhibitory RNA or mimetic specific for MIF mRNA in the mammal.

9: The method of claim 1, wherein the mammal has or is at risk for having diabetes, impaired glucose intolerance, stress hyperglycemia, metabolic syndrome, and/or insulin resistance.

10: The method of claim 1, wherein the mammal is a rodent.

11: The method of claim 1, wherein the mammal is a human.

12-13. (canceled)

14: A method of evaluating whether a compound is useful for preventing or treating type 1 diabetes, the method comprising
(a) determining whether the compound inhibits a macrophage migration inhibitory factor (MIF) in a mammal, then, if the compound inhibits the MIF,
(b) determining whether the compound inhibits development of type 1 diabetes.

15: The method of claim 14, wherein step (b) is performed by evaluating the effect of the compound on proliferation of splenic lymphocytes in the mammal.

16: The method of claim 14, wherein the compound is a protein.

17: The method of claim 16, wherein the protein comprises an antibody binding site.

18: The method of claim 14, wherein the compound is a nucleic acid or mimetic.

19: The method of claim 18, wherein the nucleic acid or mimetic is an antisense, a ribozyme, an aptamer, or an interfering RNA.

20: The method of claim 14, wherein the compound is an organic molecule less than 1000 Dalton.

21. (canceled)

22: A kit comprising
(a) a pharmaceutical composition comprising the agent used to inhibit MIF in claim 1, and
(b) instructions for administering the composition to the mammal,
wherein the mammal has type 1 diabetes or is at risk for type 1 diabetes.

23-25. (canceled)


SEQUENCE LISTING (3 sequences)

SEQ ID NO: 1 - DNA, 561 bp, Human
accacagtggtgtccgagaagtcaggcacgtagctcagcggcggccgcggcgcgtgcgtctgtgcctctgcgcgggtctcctggtccttctgccatcatgccgatgttcatcgtaaacaccaacgtgccccgcgcctccgtgccggacgggttcctctccgagctcacccagcagctggcgcaggccaccggcaagcccccccagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacagggtctacatcaactattacgacatgaacgcggccaatgtgggctggaacaactccaccttcgcctaagagccgcagggacccacgctgtctgcgctggctccacccgggaacccgccgcacgctgtgttctaggcccgcccaccccaaccttctggtggggagaaataaacggtttagagact

SEQ ID NO: 2 - DNA, 547 bp, Mouse
ggcttgggtcacaccgcgctttgtaccgtcctccggtccacgctcgcagtctctccgccaccatgcctatgttcatcgtgaacaccaatgttccccgcgcctccgtgccagaggggtttctgtcggagctcacccagcagctggcgcaggccaccggcaagcccgcacagtacatcgcagtgcacgtggtcccggaccagctcatgacttttagcggcacgaacgatccctgcgccctctgcagcctgcacagcatcggcaagatcggtggtgcccagaaccgcaactacagtaagctgctgtgtggcctgctgtccgatcgcctgcacatcagcccggaccgggtctacatcaactattacgacatgaacgctgccaacgtgggctggaacggttccaccttcgcttgagtcctggccccacttacctgcaccgctgttctttgagcctcgctccacgtagtgttctgtgtttatccaccggtagcgatgcccaccttccagccgggagaaataaatggtttataagagaaaaaaaaaaaaaaaaa

SEQ ID NO: 3 - DNA, 551 bp, Rat
gggtcacgtagtcaggtcccagacttgggtcacaccgcacttaacaccgtcctccggccgtcgttcgcagtctctccgccaccatgcctatgttcatcgtgaacaccaatgttccccgcgcctccgtgccagaggggtttctctccgagctcacccagcagctggcgcaggccaccggcaagccggcacagtacatcgcagtgcacgtggtcccggaccagctcatgacttttagtggcacgagcgacccctgcgccctctgcagcctgcacagcatcggcaagatcggtggcgcccagaaccgcaactacagcaagctgctgtgcggcctgctgtccgatcgcctgcacatcagcccggaccgggtctacatcaactattacgacatgaacgcagccaacgtgggctggaacggttccaccttcgcttgagcccgggcctcacttacctgcaccgctgttcttcgagtcttgctgcacgccccgttctgtgtttatccacccgtaatgatggccaccttccggtcgggagaaataaatggtttgagacca